(19) United States
(12) Patent Application Publication
(10) Pub. No.: US 2021/0125136 A1
(43) Pub. Date: Apr. 29, 2021

(54) System and Method for Coordination of Implant Procedures

(71) Applicant: Helia Care, Inc., Scottsdale, AZ (US)

(72) Inventor: Grant Siders, Scottsdale, AZ (US)

(21) Appl. No.: 16/605,763

(22) PCT Filed: Apr. 19, 2018
(86) PCT No.: PCT/US18/28439

§ 371 Date: Oct. 16, 2019

Related U.S. Application Data
(60) Provisional application No. 62/487,299, filed on Apr. 19, 2017.

(51) Int. Cl. G06Q 10/08; G06Q 10/06; G06Q 30/06; G16H 40/40

(52) U.S. Cl. CPC G06Q 10/08; G16H 40/40; G06Q 30/06; G06Q 10/0631

(57) ABSTRACT

A medical implant data coordination platform is implemented on a system of specially configured computing devices. The platform has implant device providers, field agents of the device providers, surgical facilities, and physicians as registered users. The platform facilitates rapid, automated processing of electronic information in order to reconcile purchase, supply, and use of the implant devices and other surgical products in a surgical procedure at the facility, by receiving an electronic requisition order from the field agent before the field agent leaves the facility, validating the requisition order against a pricing agreement between the device provider and the facility, and generating and dispensing, electronically to the appropriate devices of registered users, transaction documents such as purchase orders and invoices. The platform may receive product information from the device provider, and offered pricing information from the facility, and may execute matching algorithms to produce the pricing agreement.

### CROSS-REFERENCE TO RELATED APPLICATIONS

This application is a National Stage filing based upon PCT App. Ser. No. PCT/US2018/028439, filed on Apr. 19, 2018, which claimed the benefit of U.S. Prov. Pat. App. Ser. No. 62/487,299, filed under the same title on Apr. 19, 2017, and incorporated fully herein by reference.

### BACKGROUND OF THE INVENTION

The subject matter disclosed herein relates generally to electronic medical procedure coordination systems and methods, and, more particularly, to computer-implemented systems and methods that provide a platform that interfaces with the entities involved in a medical implant procedure to coordinate pricing, equipment selection, invoicing, invoice reconciliation, and other phases of the implant procedure.

Still today, healthcare in the United States and other countries is largely managed on paper—that is, processes ranging from product ordering and inventory to billing and reconciliation involve printed and handwritten pieces of paper changing hands, perhaps multiple times, and causing inefficiencies related to lost, buried, and unreadable documents, incompatible documentation systems, transit time for paper orders, etc. Furthermore, even when processes become digitized, they typically do not get more sophisticated than spreadsheet tracking, email exchanges, and other basic methods of electronic data management that are not accurate, efficient, homogenous (or even standardized), or centralized.

The complete process of implanting a medical device in a patient, from the initial selection of devices and materials to the reconciliation and payment of all incurred costs to all involved entities, is particularly slowed by archaic information management. In a basic case, the process involves five entities: the implant device (“implant”) manufacturer, the hospital where the procedure takes place, the doctor performing the surgery, a field agent attending the surgery and advising on needed products, and the patient's insurer. Typically, the first step is for the hospital and the manufacturer to agree on a pricing matrix that sets the expected price of each implant and associated product to be provided by the manufacturer to the hospital. However, the hospital does not purchase a set amount of the products and keep an inventory thereof. Rather, the field agent orders and maintains an inventory of the manufacturer's products in advance of the surgery.

The field agent may bring the designated implant and associated products to a particular surgery. More commonly though, due to variations in patient characteristics, procedures, and physician preferences, the field agent brings a selection of implants and products to the surgery; only some of the implants and products may actually be used. The field agent records, by hand on a hospital form, the products used during the surgery and the price thereof, and delivers the form to hospital administration for processing. The field agent also sends this information to the manufacturer, which pays the field agent according to the products used and price charged.

The hospital administration reconciles the field agent's form with the relevant pricing matrix, as well as with data describing what is expected for the surgery performed, if any such data is available. If the form is reconciled, the hospital generates a purchase order listing the used products; this purchase order is sent to the manufacturer. The manufacturer reconciles the purchase order against its own records, such as the pricing matrix and the information received from the field agent, and sends an invoice to the hospital. The hospital then generates and sends an invoice for the insured products/services to the insurer for payment. Once the insurer pays its invoice, the hospital can pay the manufacturer's invoice.

Depending on the entities involved, some or all of these steps are at least partially executed on paper, with forms being completed and reviewed manually. At best, some electronic records are used, but these are neither substantively harmonized nor made easy to access according to a common operating procedure. Moreover, false, faulty, and/or inconsistent information can be introduced through any step in this process, and various steps can be stalled by a lack of human resources, such as understaffing of hospital administration. Existing hospital inventory management systems have been unsuccessful at overcoming these issues to enable accurate tracking of implant usage. It would be desirable to provide systems and methods that implement a data coordination platform for implant procedures that can be used by entities acting in a procedure to increase accuracy and reduce processing time of information needed to complete the implant procedure.

### BRIEF DESCRIPTION OF THE DRAWINGS

The present invention will become more apparent from the detailed description set forth below when taken in conjunction with the drawings, in which like elements bear like reference numerals.

FIG. 1 is a schematic diagram of an existing manual system and method for coordinating surgical procedure data and reconciling implant usage details for payment;

FIG. 2 is a schematic diagram of an exemplary environment for a data coordination platform in accordance with the present disclosure;

FIG. 3 is a flowchart illustrating an exemplary method of coordinating surgical procedure data in accordance with the present disclosure; and

FIG. 4 is a diagram of a process for matching a facility's pricing matrix with a device providers product guide.

FIG. 5 is a block diagram depicting functional components of a system including hardware computing devices configured to implement an implant data coordination platform in accordance with the present disclosure.

### DETAILED DESCRIPTION OF THE INVENTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures. The figures depict selected embodiments and are not intended to limit the scope of embodiments of the invention. Skilled artisans will recognize the examples provided herein have many useful alternatives and fall within the scope of embodiments of the invention.

The following description refers to elements or features being “connected” or “coupled” together. As used herein, unless expressly stated otherwise, “connected” means that one element/feature is directly or indirectly connected to another element/feature, and not necessarily mechanically. Likewise, unless expressly stated otherwise, “coupled” means that one element/feature is directly or indirectly coupled to another element/feature, and not necessarily mechanically, such as when elements or features are embodied in program code. Thus, although the figures depict example arrangements of processing elements, additional intervening elements, devices, features, components, or code may be present in an actual embodiment.

The invention may be described herein in terms of functional and/or logical block components and various processing steps. It should be appreciated that such block components may be realized by any number of hardware, software, and/or firmware components configured to perform the specified functions. For example, an embodiment may employ various integrated circuit components, e.g., memory elements, digital signal processing elements, logic elements, diodes, look-up tables, etc., which may carry out a variety of functions under the control of one or more microprocessors or other control devices. Other embodiments may employ program code, or code in combination with other circuit components.

In accordance with the practices of persons skilled in the art of computer programming, the present disclosure may be described herein with reference to symbolic representations of operations that may be performed by various computing components, modules, or devices. Such operations may be referred to as being computer-executed, computerized, software-implemented, or computer-implemented. It will be appreciated that operations that can be symbolically represented include the manipulation by the various microprocessor devices of electrical signals representing data bits at memory locations in the system memory, as well as other processing of signals. The memory locations where data bits are maintained are physical locations that have particular electrical, magnetic, optical, or organic properties corresponding to the data bits.

As non-limiting examples unless specifically indicated, any database or data store described herein may comprise a local database, online database, desktop database, server-side database, relational database, hierarchical database, network database, object database, object-relational database, associative database, concept-oriented database, entity-attribute-value database, multi-dimensional database, semi-structured database, star schema database, XML database, file, collection of files, spreadsheet, or other means of data storage located on a computer, client, server, or any other storage device known in the art or developed in the future. File systems for file or database storage may be any file system, including without limitation disk or shared disk, flash, tape, database, transactional, and network file systems, using UNIX, Linux, Mac OS X, Windows FAT or NTFS, FreeBSD, or any other operating system.

The various aspects of the invention will be described in connection with providing a data coordination platform that enables entities involved in a medical implant procedure to digitize, reconcile, and otherwise manage information related to the implant procedure. That is because the features and advantages that arise due to the invention are well suited to this purpose and overcome the drawbacks of previous systems for addressing the same problem of surgical procedure data management. However, it should be appreciated that the invention is applicable to other procedures and to achieve other objectives as well.

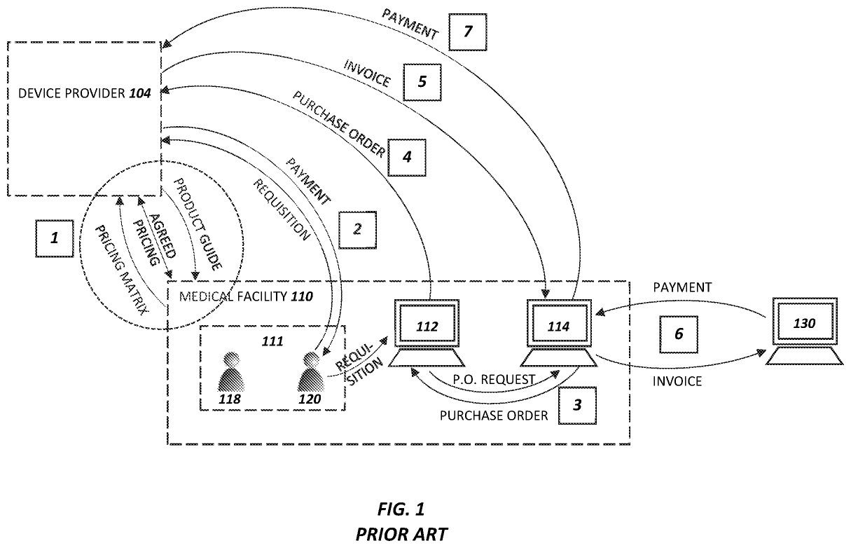

FIG. 1 illustrates the present (i.e., before this disclosure) environment in which entities involved in a medical implant procedure exchange data about the procedure in order to record products used in the surgery and process payments for the used products. As described above, this procedure, from the initial selection of devices and materials to the reconciliation and payment of all incurred costs to all involved entities, is slowed by archaic information management. In a basic case, the process involves five entities: the implant device provider 104, which may be a device manufacturer, a wholesaler or other type of vendor, or another entity that makes available the products needed for the surgery; the hospital or other medical facility 110 where the procedure takes place, a physician 118 performing the surgery (i.e., in an operating room 111); a field agent 120, such as a sales representative of the device provider 104, attending the surgery and advising on needed products; and the patient's insurer or another payor 130 responsible for paying all or part of the procedure costs.

At some point before an implant or other device provided by the device provider 104 can be used at the medical facility 110, the facility 110 and the device provider 104 may agree on a pricing matrix that sets the expected price of each implant and associated product to be provided by the device provider 104 to patients in the facility 110. The pricing matrix is, essentially, a hierarchical set of categories; most typically arranged by anatomical region at the top level of the hierarchy, the matrix then may then expand to one or more anatomical sub-regions. At a desired level of specificity regarding the body part, the matrix expands to particular surgical procedures that apply to the body part; the surgical procedures may have known standard identifiers, such as DRG codes, associated therewith. Then, each procedure may be expanded to a list of implants and associated products to be used in the surgical procedure. The implants and products may be associated with product numbers and other identifiers, as well as an agreed-upon price for the product.

Many different factors impact the determination of the data in the pricing matrix. For example, one or more physicians 118 authorized to practice at the facility 110 may require or forbid certain products, which can affect the listing and price of those products and other products. The overarching objective, however, is to reconcile the facility's 110 base matrix of the types of surgical procedures and products the facility 110 will pay for with the device provider's 104 catalog or product guide, which may include the device provider's 104 base prices for its products. This process historically has proven to take between six and 12 months to complete.

The field agent 120 is an employee or independent contractor of the device provider 104 who has a relationship with the facility 110 and/or the physician 118. Once the device providers 104 devices have been accepted for use at the facility 110, the field agent 120 may be authorized to personally attend surgeries that use the devices. The field agent 120 orders and maintains an inventory of the device provider's 104 products in advance of the surgery. In some embodiments, the field agent 120 has the requisite devices in his/her possession and brings them to the operating room 111 on the day of the surgery; in other embodiments the field agent 120 merely coordinates the delivery of the devices and products to the operating room 111. The field agent 120 attends the surgery, possibly assisting and making recommendations for the use of the products.

The parameters of each surgery indicate generally which products, and how many of each, will be needed. These parameters may be set by the hospital and/or the implant manufacturer; useful generalized values for the parameters with respect to a particular surgical procedure may be difficult to ascertain due to differences between the individual cases in which the surgical procedure is applied. Non-limiting examples of conditions affecting an accurate characterization of a surgical procedure include: different hospitals using the manufacturer's devices in differently configured facilities; a particular hospital having differently configured operating rooms; different doctors qualified to perform the surgery having different preferences for implants and products; and, of course, the biological characteristics of the patient being operated on. When it is difficult to determine which and how many products will be used, even with a mandatory pricing matrix it can be difficult or impossible to estimate the cost of the surgery.

Another factor complicating a cost determination in advance is the field agent 120, who is typically paid by the device provider 104 in accordance with the volume of products used. The field agent 120 may bring surplus units of the designated implant and associated products to a particular surgery, and might legitimately (e.g., a product becomes unsterile and must be replaced during the surgery) or illegitimately (e.g., the field agent marks an unused product as used) use extra units. The field agent 120 records, by hand on a hospital form, the products used and the price thereof and delivers the form to facility administration for processing. The field agent 120 also files this report with the device provider 104, which pays the field agent 120 according to the products used and price charged.

The facility administration staff manually reconciles the field agent's 120 form with the relevant pricing matrix, as well as with data that indicates that the report of products used is commensurate with what is expected for the surgery performed, if any such data is available. A second internal check is made between the facility's 110 medical and accounting departments. For example, a medical assistant may request (manually or using a device 112) a purchase order for the products on the validated form from an employee in the accounting department, and if the employee can verify (manually or using a device 114) that the information is correct, the employee may generate (manually or using the device 114) a purchase order listing the used products and give or send it back to the medical assistant. This purchase order is sent by the medical assistant to the device provider 104. The device provider 104 reconciles the purchase order against its own records, such as the price matrix and the report filed by the field agent 120, and sends an invoice to the accounting department of the facility 110. The accounting employee then generates (manually or using the device 114) a procedure invoice, including the costs on the invoice as well as other costs incurred in the surgical procedure, and sends the procedure invoice to the payor 130. The payor 130, which may be an insurer, then reconciles the received invoice with its own records indicating whether and how much it will pay for the line items of the invoice, and may coordinate corrections to the invoice with accounting 114 before paying the invoice.

As explained above, in present systems these steps are at least partially executed on paper, with forms being completed and reviewed manually. Additionally, those steps that include some automation and/or digitization of information produce electronic records, such as spreadsheets, electronic documents, emails with attachments, etc., that are neither substantively harmonized nor made easy to access according to a common operating procedure. Moreover, false, faulty, and/or inconsistent information can be introduced, and unauthorized use of products may be obfuscated or overlooked, at any step in this process, and various steps can be stalled by a lack of human resources, such as understaffing of hospital administration. Delays are common, especially when information is maintained by hand and frequent exchanges of the same order are required to reconcile the information. Presently the average time from the day the surgery is performed until all parties have been paid and the procedure is complete is between three and six months, and can be much longer. Many cases generate a payment gap for one or both of the manufacturer and the hospital, where the entity's costs related to the procedure are due to be paid before (sometimes months before) the entity receives payment from the hospital or insurer, respectively. A sixth entity—a lender—may enter the procedure, loaning interest-bearing funds to cover the shortfall. Considering the thousands of implant procedures performed by a hospital each year, the associated costs of financing the payment gap are a major contributor to hospital expenses.

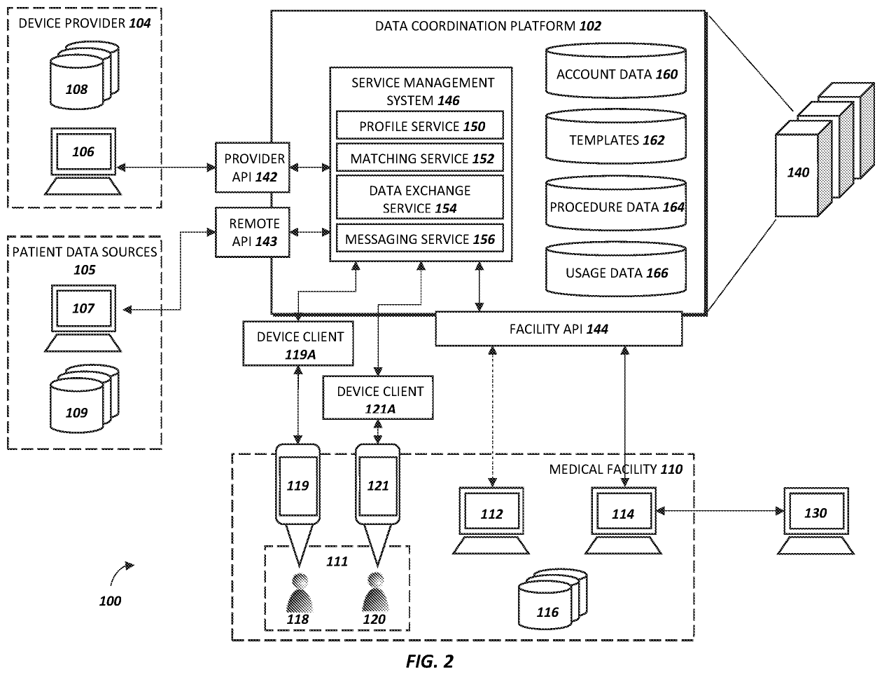

FIG. 2 illustrates an exemplary environment 100 in accordance with the present disclosure, in which entities involved in a medical implant procedure use their computing devices to access a data coordination platform 102 that enables the entities to electronically interact with each other to determine the equipment to be used in an implant surgery and to invoice and reconcile the costs of such equipment. The data coordination platform 102 may include and/or be implemented using computing hardware and software that provides computational resources of at least one physical computing device 140 to one or more user devices that interface with the platform 102 to execute services and store and access data. The data includes data related to a particular surgical procedure and the services are used to coordinate the data between entities. The platform 102 may be implemented in the cloud (on one or more virtual machines), on a remote server, or across multiple servers communicating with each other over an electronic network. Additionally or alternatively, the platform 102, or parts thereof, may be implemented on one or more user devices, such as a mobile phone or tablet, using the hardware and software interfaces of the mobile device, including communicating with a user of the mobile device via sound or visual displays, and communicating with objects on remote devices, such as data stores of the platform 102, via the internet or a cellular network. For example, a user of a mobile device may install on the mobile device an application, software service, firmware, or other device client that serves as the interface to the platform 102 or otherwise accesses the platform 102 services.

The entities described above with respect to FIG. 1 may be enabled to use the platform 102 and exchange data with the other entities as described herein. In comparison to existing systems that use a combination of manual and uncoordinated electronic processes, the platform 102 and associated systems provide significantly improved efficiency, accuracy, and simplicity in entering, validating, resolving, and processing data from the multiple entities in order to complete transactions involving medical implants. The device provider 104 may access the platform 102 using one or more user devices 106 that store or connect to an application programming interface (API) 142 that provides access to the platform 102. The user devices 106 can send data, such as implant and product data stored in one or more local data stores 108, to the platform 102 for processing, and can send data (e.g., messages) through the platform 102 to other entities, as described further below. Similarly, user devices 107 of one or more patient data sources 105 storing relevant patient data in remote databases 109 may access, or be accessed by, the data coordination platform 102 via a remote API 143; this connection allows the exchange of patient data produced or stored by either system 102, 105 with the other to improve patient outcomes by keeping patient data up-to-date. Also similarly, user devices for the medical assistant 112 and the facility accounting 114 may use a facility API 144 to access the platform 102 and use the platform services to manage data, such as pricing data, facility data, procedure data, patient data, etc., stored in one or more local data stores 116. As described above, mobile devices may also be used with the platform 102; thus, mobile users such as the physician 118 and field agent 120 may use their respective mobile devices 119, 121 to access the platform 102, such as through device clients 119A, 121A installed on the respective devices 119, 121.

Any of the APIs 142-144, device clients 119A, 121A, and other applications and user interfaces for accessing the platform 102 may be implemented in any suitable software framework, such as MICROSOFT .NET, Amazon Web Services, ActiveX, SOAP, and the like. In some embodiments, each API may be embodied in a web application that is operable in, and/or accessible by, an internet browser installed on the various user devices 106, 112, 114, 119, 121. The web application may provide a graphical user interface using any suitable internet technology, such as HTML. In one embodiment, all messages and other data exchanges pertaining to data that is managed by the platform 102, including messages between entities, may be passed through the platform 102 via the corresponding APIs and device clients, which may perform one or more of input validation, error handling, and interfacing with particular platform 102 services. Facilitating inter-entity and entity-to-service messaging through the APIs allows for standardization of message formatting and data access.

In some embodiments, a device client 119A, 121A may be a hardware or software component, as is suitable for the device on which it is installed; a device client may be installed on all user devices accessing the platform 102 in some embodiments. Suitable devices include any device that can be configured to transmit information about its state or receive input that modifies its state. Examples of such devices include, without limitation: personal computing devices such as desktops, laptops, tablet computers, mobile phones, digital media players, and the like; home or office audio or video equipment, such as televisions, projectors, theater components, recording or playback devices, and the like; dedicated servers, such as application, communication, mail, database, proxy, fax, file, media, web, peer-to-peer, standalone, software, or hardware servers, which may use any server format known in the art or developed in the future (possibly a shared hosting server, a virtual dedicated hosting server, a dedicated hosting server, or any combination thereof); monitoring systems, such as home security systems, thermostats, vehicle status monitors, infant monitors, and the like; wearable devices, such as watches, goggles, bracelets, devices implanted into cloth, and the like; and biological implants, such as pacemakers, catheters, and the like. Suitable devices may further include software-based or pure-software devices, including, without limitation: cloud computing frameworks, such as AMAZON ELASTIC COMPUTE CLOUD, MICROSOFT WINDOWS AZURE, and the like; search engines; social networks; and email services. The installation of the device client may authorize the device in the platform 102, or a user may separately authorize the device. Once installed, the device client may coordinate local device resources for access by the platform 102. Such coordination may include providing, to the platform 102, access to all or a subset of the user's documents, photographs, device settings, applications, usage authorizations, and other information stored on the local device, as well as control of all or a subset of the device's equipment, such as video camera, speakers, sensors, and the like. Such access may depend on permissions set by the user.

The platform 102 may implement or support a service management system 146 that coordinates invocations of various services of the platform 102. Such coordination may include acquiring and managing computing resources of the physical computing devices 140, providing interfaces to local and/or remote data stores, receiving, formatting, parsing, processing, and delivering requests between requesting and target devices, and the like. FIG. 2 illustrates some exemplary services that can be made accessible to user devices according to preset permissions, such as those corresponding to an authentication scheme. For example, the service management system 146 may include or communicate with an authorization system that checks credentials (e.g., username/password combination, secure socket layer certificate, private key, etc.) presented by a user device, and the service management system 146 may grant certain access to certain services and to certain data stores according to the user authentication/authorization.

A profile service 150 may receive data submitted by a user device in order to generate and/or maintain a user profile associated with an entity using the system. The profile service 150 may, for example, store this data in an account data store 160 in accordance with any suitable data structure or data record configuration. Various entities may have one or more user profiles associated therewith, such as a device provider 104, one or more particular users associated with the device provider 104 (e.g., various employees of the device provider 104, or offices/departments/divisions of the device provider 104), a medical facility 110, employees or departments of the medical facility 110 (e.g., a medical assistant 112 or an accounting department 114), a physician 118, and a field agent 120. A user profile may have various permissions associated therewith, such as restrictions on which services can be used and which data stores can be accessed. In various embodiments, the user profile of an entity includes information that can be used by other services to coordinate data related to one, some, or all implant procedures with the relevant entities. Some types of such information may be common to all entities and to all user profiles, such as identifying information (e.g., name and address of hospital), contact information, and the like.

Other information may be particular to the type of entity. Non-limiting examples for a medical facility 110 include data, such as raw text or files, describing the facility's resources, including physicians licensed to practice there, operating rooms and equipment, facility certifications, and the like. In some embodiments, the information may include one or more price matrices that the facility 110 uses to set terms with a device provider. The price matrices may include existing matrices, such as a default price matrix and/or existing price matrices agreed upon with a device provider (e.g., device provider 104 or another manufacturer, supplier, etc.) that has an existing supply relationship with the facility 110. In various examples, a facility's 110 price matrices may be represented by a single matrix, or by a set of matrices each pertaining to a discrete anatomical region, and/or by matrices each pertaining to a particular category with an anatomical region. The profile service 150 may be configured to intake these existing matrices in any format (e.g., an EXCEL spreadsheet, an ADOBE PDF, a set of values manually entered into the API, etc.) and convert them into a standard format in which they are stored in the account data store 160. Thus, in one embodiment, the profile service 150 is configured to present a user interface via any of the APIs/device clients, in which the user is prompted to enter as much information as possible about the existing matrix and then upload the corresponding file(s). For example, the matrix may be determined by analyzing an uploaded document, such as a spreadsheet, a scan of a hard-copy matrix, and the like, to identify and extract products, product codes, categories, prices, and other data expected to be in the matrix. The system may receive user input to guide the analysis, such as in response to a prompt for the user to identify the classifications used in the matrix. Additionally or alternatively, the system may perform optical character recognition and/or machine learning algorithms to the uploaded files to extract the relevant matrix information.

In some embodiments, the profile service 150 may allow the user to create a new pricing matrix for use with the platform 102, starting from a pricing matrix template selected automatically or by the user from a plurality of templates stored in a template data store 162. Each template may correspond to a commonly-used format for pricing matrices; the user may be enabled to select the template that corresponds to the format the facility 110 presently uses, or the template closest thereto. The user may then enter the data from the locally-stored (e.g., in the data stores 116) pricing matrix, manually and/or by uploading files to the platform 102. Advantageously, the profile service 150 may then store the pricing matrix using a known data structure that is easiest to associate with the device provider's 104 data as described below. Some facilities 110 may only need to enter a base pricing matrix once and reuse it many times without having to reenter any information.

Likewise, the user account data for a device provider 104 may include data, such as raw text and files, describing the available implants, devices, products, etc. Such data may include certifications and disclosures (e.g., 501(k) statements) for each product, product specifications, testimonials and analytical data, etc. In particular, the data may include a catalog or product guide listing essential product data, such as item number and name, price, diagnosis related group (DRG) code(s), associated surgical procedures, associated products, etc. The profile service 150 may be configured to intake the product guide in any format (e.g., an EXCEL spreadsheet, an ADOBE PDF, a set of values manually entered into the API, etc.) and convert it into a standard format in which it is stored in the account data store 160. Thus, in one embodiment, the profile service 150 is configured to present a user interface via any of the APIs/device clients, in which the user is prompted to enter as much information as possible about the product guide and then upload the corresponding file(s). The product guide information may then be extracted from the provided files as described above. In some embodiments, only the facility 110 may enter product categories, and only the device provider 104 may enter products.

In some embodiments, the profile service 150 may allow the user to create a new product guide for use with the platform 102, starting from a product guide template selected automatically or by the user from a plurality of templates stored in the template data store 162. Each template may correspond to a commonly-used format for product guides; the user may be enabled to select the template that corresponds to the format the device provider 104 presently uses, or the template closest thereto. Additionally or alternatively, the system may determine that the selected pricing matrix template is associated with a particular product guide template that facilitates matching between the templates, and the system may select or recommend the particular product guide template. The user may then enter the data from the locally-stored (e.g., in the data stores 108) product guide, manually and/or by uploading files to the platform 102.

The profile service 150 may further be enabled to organize the product guide into any suitable categorical hierarchy, such as one based on anatomical regions as described above. Thus, in some embodiments, a device provider 104 that does not use a hierarchy in its products or product guide may adopt a hierarchy into the version of the product guide stored for use on the platform 102; in particular, the device provider 104 may adopt the hierarchy or other categorization used by the facility 110, which facilitates matching. In another embodiment, where the product guide uses a different hierarchy from those made available by the profile service 150, the profile service 150 may, via user interfaces, enable the device provider 104 to identify correlations between its native product guide hierarchy and the hierarchy used by the facility 110.

Using uploaded files and user input, the profile service 150 may store (e.g., in the account data 160 associated with the device provider 104) the product guide into one or more predetermined data structures that are configured to be associated directly with the facility's 110 platform-configured pricing matrix data structure(s). For example, a product guide data structure and a pricing matrix data structure may both include a plurality of category definitions each associated with an anatomical region, and each having a corresponding category definition in the other data structure; each category definition may then include a plurality of sub-category definitions each associated with a more specific anatomical region, and each sub-category definition may include a plurality of procedure definitions each associated with a procedure. Each procedure definition may then include one or more product definitions for products to be used with the procedure. In the pricing matrix data structure, a product definition may simply be a data structure indicating the expected parameters, such as product name, product description, quantity needed for the procedure, price, and approval flag. In the product guide data structure, the product definition may have the same structure, but there may be multiple records each containing the actual data for a product entry in the product guide. In this example, the fields of the product guide and pricing matrix data structures have a direct (i.e., one-to-one) correlation with each other that is stored in memory of the service management system 146; the procedure to be performed is the primary identifier for correlating product entries with the procedures that can be performed at the facility 110. In another example, the pricing matrix data structure may have the configuration of the previous example, while the product guide data structure uses product definitions instead of procedure definitions. The product guide's product definition may identify a plurality of procedures in which the associated product can be used; the system may associate (e.g., for matching purposes as described below) this product definition with each of the procedure definitions in the pricing matrix that define one of the procedures identified in the product definition.

A matching service 152 may be implemented to connect device providers 104 with facilities 110 that need devices. The matching service 152 may operate partially or completely autonomously on the pricing matrices and product guides stored in the account data store 160 to identify “matches” and potential matches between the products offered by a device provider 104, and the products needed at a facility 110 to perform procedures. The matching service 152 may perform comparisons of the data in some or all of the product entries in the product guide data structure against some or all of the procedure definitions; a first level of comparisons may determine which products in the product guide are needed in procedures performed at the facility 110, and a second level of comparisons may determine whether the device provider's 104 price for a needed product is in line with the price the facility 110 is willing to pay. In some embodiments, the matching service 152 may perform some or all of the comparisons based on one or more default or user-provided thresholds for defining matches. A match may be produced when, for example, the device provider 104 offers at least a threshold percentage of the products that appear on the facility's 110 pricing matrix. Additionally or alternatively, a match may indicate that a threshold amount of the prices requested by the device provider 104 align with the prices offered by the facility 110. A potential match may be found for a product when the price requested by the device provider 104 is within a threshold percentage of the price offered by the facility 110 (indicating some degree of negotiation between the parties may bring the prices into alignment and create a match).

The matching service 152 may produce a match result data structure comprising entries for each of the products in an identified match. In some embodiments, these match results may be stored (e.g., in the account data 160) for later processing. In other embodiments, the match results may be sent or otherwise presented to one or both of the device provider 104 and the facility 110, such as in a user interface that enables a user to perform actions related to the matches. For example, a hospital administrator can review and accept or reject some or all of the matches; accepting the matches may cause the matching service 152 or another service to add the product to a data structure representing a supply agreement under which the device provider 104 agrees to make products available at the facility 110 at the agreed-upon price. In another example, a manufacturer administrator can review the matches, select a near-match where the requested and offered prices are within a negotiation threshold but do not match, and enter a counter-offer price that is transmitted to the facility 110 administrator for consideration. The system may additionally or alternatively be configured to accept the price match automatically, and further to enable the agent to indicate prior to the execution of the matching algorithm that none, some, or all of the price matches can be automatically accepted.

This automated matching and partial matching may be completed in seconds or minutes, with little additional work needed once a match or partial match is produced, in order to establish a facility/provider pricing agreement. In some embodiments, creating a user account for a device provider 104 or a facility 110 adds the new user account to a group of user accounts that the matching service 152 parses to find matches, and the user can opt-out of the group. In other embodiments, the new user account is not automatically added and the user must request that the account be added, essentially enabling matching for the user account. Either way, the platform 102 becomes the point of first contact between the device provider 104 and the facility 110. The matching service 152 may store data describing matches, partial matches, and non-matches for each user account in the account data store 160 for later retrieval. The matching service 152 may also store data describing the agreement reached between the provider and facility, such data including identifiers for each party and a pricing matrix containing the products and prices agreed upon. An agreement data structure may further include parameters describing proper use of an agreed-upon product. For example, an agreed-upon product entry in the data structure may include the quantity of the product expected to be used in an associated procedure (this value may be stored in the pricing matrix or entered by the facility or by the device provider after a match is identified).

A data exchange service 154 may coordinate the exchange of information related to a surgical procedure between entities using the platform 102. The service 154 and other services of the service management system 146 may begin administration of a particular surgical procedure upon scheduling of the procedure, once the procedure is initiated, during the procedure, or after the procedure, in various embodiments; such administration may be initiated at a predetermined time (e.g., at the beginning of a workday or the scheduled start time of a procedure) or in response to a triggering event (e.g., receipt of a requisition order or another document or message). In some embodiments, the data exchange service 154 may restrict or prevent performance of a new surgical procedure at the facility 110 until the platform 102 has generated and stored (an) agreement(s) (i.e., containing agreed-upon pricing matrices) between one or more device providers 104 and the facility 110 to supply all of the products needed to perform the procedure; the data exchange service 154 may further verify that the devices and products to be used in the procedure are listed in one of the agreed-upon pricing matrices. In one embodiment, the new surgical procedure may be initiated according to a surgery schedule maintained on the platform 102 (e.g., via storage of scheduling data in the account data store 160) by the facility 110 and/or by one or more physicians 118 working at the facility 110. In other embodiments, the new surgical procedure may be initiated upon receipt by the data exchange service 154 of one or more requisition orders for devices and/or products used in the surgical procedure, as described further below. The service 154 may begin administering the surgical procedure on the platform 102 when a first requisition order is received, even if some of the necessary devices/products are not listed in the order; or, the service 154 may only allow the surgical procedure to be initiated (i.e., administered on the platform 102) when all of the products have been requisitioned.

A requisition order is produced on the platform 102 by the field agent 120 using his/her user device 121. For example, the device client 121A may display a user interface on the user device 121 enabling the field agent 120 to identify the products that the physician 118 used in the surgical procedure. The user interface may be configured to receive other data related to the products, such as the quantity used. In some embodiments, the data exchange service 154 and/or the device client 121A may configure the user interface so that the field agent 120 cannot enter product usage data that deviates from corresponding information (i.e., from the agreement data structure, agreed-upon pricing matrices, procedure data 164, and the like) describing the agreed-upon use and cost with respect to a selected product and the particular procedure. For example, the field agent 120 may select the surgical procedure being performed (or the procedure may be automatically selected based on scheduling data and/or other facility 110 information), and the user interface may be updated to display a list of the products (provided by the field agent's 120 associated device provider 104) and respective quantities that should have been used in the procedure. The field agent 120 may only be able to confirm that the products and proper quantities were used. In other embodiments, the field agent 120 may be able to change some of the data, such as identifying a substitute product, increasing the quantity used, or modifying the default price. Such changes may cause the user interface to display warning indicators that the usage parameters diverge from the specifications. The user input may nevertheless be accepted, and the requisition order generated from the user input may include variance flags identifying usage that is out of conformance.

Using the platform 102, the facility 110 and/or the device provider 104 can require that the field agent 120 produce the requisition order immediately and transmit it while still in the operating room 111. For example, the data exchange service 154 may have access to the location of the user device 121, such as by communicating with the device client 121A to repeatedly request and receive the GPS data of the user device 121. The service 154 may obtain geo-location data of the facility 110, by querying the account data store 160, another data store of the platform 102, or an external data store; this geo-location data may include the coordinates of the operating room 111. Alternatively, the service 154 may communicate with a proximity sensing device disposed in the operation room 111, which detects a distance of the user device 121 from the sensing device. The service 154 may generate an alert or error if it detects that the user device 121 left the operating room 111 or the facility 110 and the service 154 has not received the requisition order. Or, the service 154 may receive the requisition order together with a location indicator giving the location of the user device 121 when the requisition order was submitted.

Another way the data exchange service 154 may use the location of the user device 121 to control accuracy and timeliness of the requisition order is to limit the data elements available for selection in a user interface presented on the user device 121 based on the user device's 121 location. For example, detecting that the user device 121 is in a particular facility 110, the data exchange service 154 may only allow the field agent 120 to select products and quantities that the facility 110 or the device provider 104 has authorized for that facility 110. Further, the data exchange service 154 may not allow the field agent to change the prices of devices and products, instead presenting a static value retrieved from the agreed-upon pricing matrix. The data exchange service 154 may also communicate with the physician's device 119, in some embodiments using the device 119 location as described above, to request and receive confirmation from the physician 118 that the requisition order is accurate.

The device client 121A may send the user input to the data exchange service 154 for processing, or may itself process the user input. Processing the user input may include generating a requisition order comprising the user input arranged into structured data having a format that the data exchange service 154 may quickly and accurately parse into corresponding orders (i.e., one or more of a field agent 120 invoice, a facility 110 purchase order, a device provider 104 invoice, and a facility 110 invoice) used by the device provider 104 and the facility 110 to reconcile the product transaction. Notably contrary to prior art systems, the requisition order generated by the field agent 120 is not delivered to the medical assistant 112 or to another facility 110 employee or department tasked with validating the order before the order is processed by accounting 114. Consequently, the medical assistant 112 may not need to handle any data for processing the surgical procedure and may be omitted from the process (thus, FIG. 2 illustrates the medical assistant 112 communication with the platform 102 as optional), eliminating a potential source of error and delay. Furthermore, this functionality of the platform 102 may be used by the device provider 104 and field agent 120 even if the facility 110 is not registered with the platform 102; for example, the service 154 may parse the requisition order into an invoice but not a purchase order.

The data exchange service 154 and/or another service may be configured to parse the requisition order and determine the products used in the surgical procedure. The service 154 may validate the product usage as indicated by the requisition order, and then generate the corresponding orders if there is no erroneous or missing usage information. In some embodiments, the service 154 may identify variance flags or other data values indicating the product usage or pricing does not conform to the expected outcome of the surgical procedure. For example, a parameter value (e.g., quantity used) that was changed from the default proper value may be flagged in the requisition order data structure. In another example, the service 154 may determine the value (e.g., quantity or price) from the requisition order and compare it to a stored acceptable value (e.g., in the agreement data structure associated with the corresponding device provider 104). In some embodiments, variances may not be permitted, and the requisition order may be rejected. For example, the service 154 may cause the device client 121A to alert the field agent 120 that the requisition order must be resubmitted. In other embodiments, the service 154 may send information describing the requested variance to the appropriate entity for approval. For example, the requisition order may include a description field in which the field agent 120 explained that a screw broke during insertion and one additional screw had to be used; the service 154 may send this information to one or both of the device provider 104 and facility 110 with a request that the entity approve or deny the requested variance. In another example, the service 154 may generate orders including the modified usage data, which the corresponding entity then can approve or deny together with the rest of the order. In another example, the device provider 104 may provide a pricing agreement to the platform 102 that is made with a facility that is not registered with the platform 102; the data exchange service 154 may use the pricing agreement to validate the requisition order with respect to the device provider 104 only.

The new orders may include identifiers having a format that is known to each of the entities: for example, the orders may include an invoice number and a purchase order number that each conform to the respective entities' accounting systems. In some embodiments, the data exchange service 154 may then deliver the orders to the respective entities for validation/approval. For example, the service 154 may: generate a draft purchase order including the purchase order number, the product usage information, and the amount of the order, and deliver the purchase order to accounting 114 for approval; generate a draft invoice including the invoice number, a list of the used products, and the amount to be paid, and deliver the invoice to the device provider 104 for approval; generate an agent invoice for paying the field agent 120; and, generate a final invoice incorporating the product orders of all device providers having products used in the procedure, the final invoice to be paid by the payor 130 to the facility 110, and deliver the final invoice to accounting 114 for approval. In various embodiments, one or more suitable delivery mechanisms may be used such as an email message, a web message or other notification within a web application dashboard accessible by the relevant user devices 106, 114, and the like.

End users of the respective entities may review, modify, deny, and/or approve the received orders; when approvals are received, the data exchange service 154 may generate appropriate payment documents, such as an invoice for the facility 110 to pay the device provider 104 and/or an invoice for the payor 130 to pay the facility 110. Payment to the field agent 120 may also be generated and delivered, or the records sent to the user device 106 of the device provider 104 for aggregation (e.g., in the local data stores 108 for later payout). Generation of approved purchase orders and invoices can occur synchronously or asynchronously in various embodiments. For example, in one embodiment the service 154 may not generate the draft invoice for review by the device provider 104 until the service 154 an approval from accounting 114 of the draft (or modified) purchase order; this allows the service 154 to identify any changes to the draft purchase order and generate the draft invoice accordingly. In another example, changes by the field agent 120 to the default agreed-upon usage parameters may be prohibited; thus, the validated requisition order contains usage information upon which the device provider 104 and facility 110 have already agreed, and the service 154 may skip the draft approval process and instead generate and deliver the final orders to the entities.

Data related to processing a particular surgical procedure may be stored in various data stores for different uses. For example, the data can be stored in the account data store 160 in association with the relevant user accounts to track a history of processed surgical procedures. In another example, the data can be stored in a procedure data store 164 that aggregates data for particular surgical procedures and/or for certain devices and products, in order to refine common parameters used by the system. For example, the data can be used to determine that a certain number of units of an ancillary product are typically used in a particular surgical procedure (e.g., pedicle screws in a spinal fusion surgery). Similarly, the data may be stored in a usage data store 166 that aggregates exchanged data to determine characteristics of how the platform 102 is being used by the various entities. The data exchange service 154 or another service may deliver various aggregated data to a user interface, such as a web application dashboard or console, to enable the user to review data within a scope encompassing more than one partner entity. For example, accounting 114 of the facility 110 may use the facility API 144 to view a distributor control interface that can list all surgical procedures having unresolved product transactions. The control interface may further display data with more granularity; for example, a user click on one of the listed procedures may cause the interface to display a list of all products used or being used in the procedure, and accompanying data. The accompanying data can identify the device provider 104 and/or the field agent 120 associated with the product; thus, the user can view the products and associated requisition/payment status across multiple device providers at once.

A messaging service 156 may facilitate communications between the different entities using the system. The messaging service 156 may be configured to convert between different formats of messages, such as when sending messages from a mobile device to a desktop or web application. The messaging service 156 may also identify usage permissions of different users attempting to send messages, to determine whether the users are authorized to do so. Messages, and any other data, may be encrypted and/or sterilized in order to satisfy any applicable data security regulations, particularly with respect to health care and personally-identifying information.

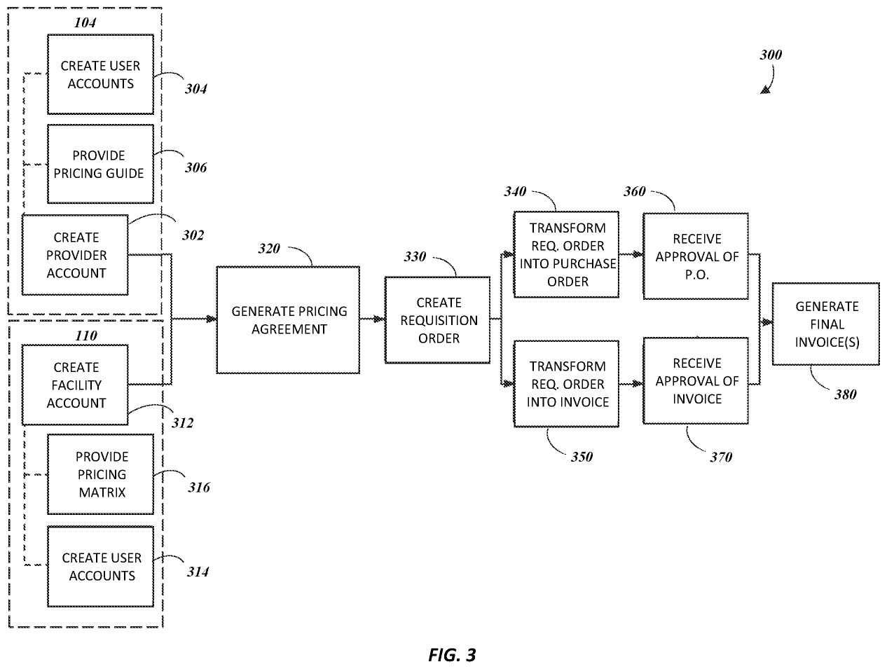

FIG. 3 illustrates an exemplary method 300 in which a system implementing the data coordination platform of the present disclosure is used to electronically reconcile claims of devices and products used in a surgery and generate one or more invoices as quickly as any users prompted to enter information can do so. The platform may first be configured to service the entities involved in the surgical procedure via a registration process of the entities. For example, various user accounts may be first created by supplying relevant information as described above. Thus, at step 302 an administrative user representing the device provider may create a user account for the company. Individual user accounts for various users associated with the company, such as accounting department employees and field agents, may also be created (step 304). Creating the user account may include providing, as described above, a product guide or catalog (step 306) describing the device provider's products in a format or structure that is usable on the platform; alternatively, a user may provide the product guide/catalog subsequent to account creation.

For example, the system may provide a user interface that can be displayed on the user's computing device, and the user can use the interface to provide the product guide by uploading one or more files and/or entering product data into the user interface in response to prompts. In some embodiments, the interface may enable the user to select, or may automatically select and provide, one or more templates such as a product guide template or a pricing matrix template as described above; the user may enter the product data using the template. In one embodiment, the template may be an existing pricing matrix of a facility 110 that is already registered with the platform. The system may receive user input from the user's computing device and generate a product guide data structure arranged in a predetermined or user-provided format and comprising a product entry for each of the products described in the user input. In one example, the system creates a product entry including a product name, description, DRG code(s), procedures using the product, requested (e.g., retail) price, and the like. The system may receive uploaded documents associated with the product, such as materials handling specifications and other spec sheets, and may store the documents in the product entry, or may store the documents in a document data store and include a reference to the storage location in the product entry.

In a similar manner, an administrative user for the medical facility 110 may create a user account for the facility (step 312), and individual user accounts may also be created (step 314). A pricing matrix or a similar data set, such as a hierarchy of surgical procedure categories together with prices offered to pay, may be provided to the platform (step 316) at or after account creation as well. For example, the system may provide a user interface that can be displayed on the user's computing device, and the user can use the interface to provide the pricing matrix by uploading one or more files and/or entering category, procedure, product, and/or offered price data into the user interface in response to prompts. In some embodiments, the interface may enable the user to select, or may automatically select and provide, one or more templates such as a pricing matrix template as described above; the user may enter the data using the template. The system may receive user input from the user's computing device and generate a pricing matrix data structure arranged in a predetermined or user-provided format and comprising a definition for each procedure described in the user input, the procedure definition including information that can be used to identify the necessary products and the associated pricing of the products (or of the overall procedure including all of the necessary products) that is the target cost of the facility 110. In one example, information related to the procedure may include DRG codes, general product categories (e.g., “spinal screws”), and/or more specific product identifiers.

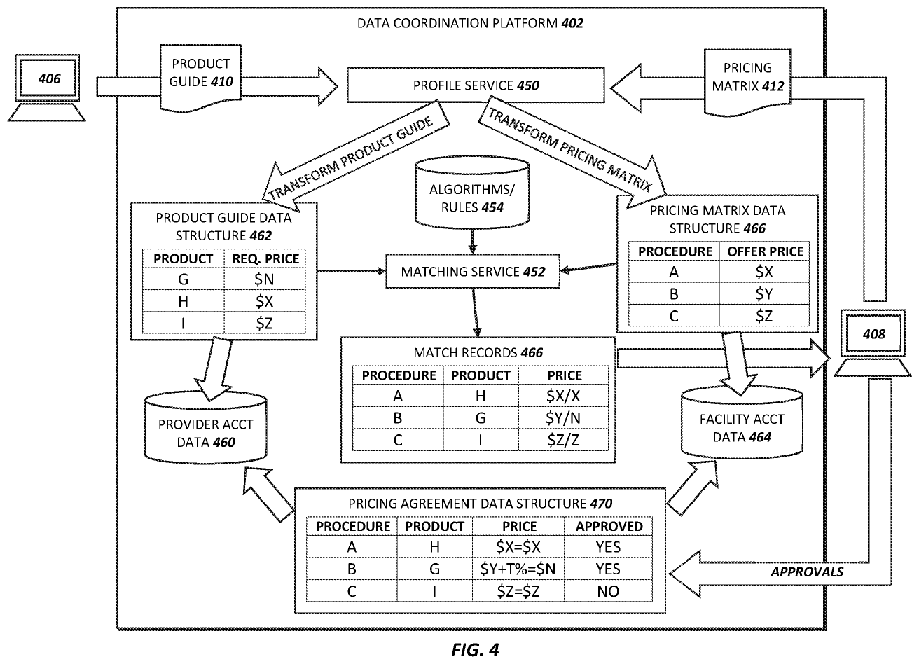

When the corresponding user accounts are created and the necessary data related to products and pricing is present on the platform, and provided the user accounts are configured to engage the matching service, at step 320 the system may generate a pricing agreement between the device provider and the facility on products and pricing. For example, the system may compare (e.g., via execution of one or more matching algorithms) the device provider's product guide and the facility's pricing matrix to identify matches upon which the entities may establish an agreed-upon pricing matrix as described above and as further detailed with respect to FIG. 4, below. As part of step 320, the system may create an initial connection between the device provider and the facility. For example, the system may generate a notification including match information and identifying information for each entity and send the notification to both entities via the data exchange service of the platform. In some embodiments, when the connection is created, the entities may use the messaging service of the platform, or may communicate outside of the platform, to negotiate the agreement. Via a combination of data entry and confirmatory inputs, the user(s) may enter the agreed-upon pricing matrix into the platform. Additionally or alternatively, the system may be configured to accept some or all of the matches automatically.

The subsequent steps may be executed for each individual surgical procedure. In the example case, the system creates a requisition order for products used in a surgery (step 330). In some embodiments, this takes place at the time of the surgery or shortly thereafter, in accordance with the configuration of the platform services as described above. In other embodiments, the system may begin to collect data related to a particular surgical procedure in advance of the actual surgery; non-limiting examples of such data that the platform may use to improve data coordination include scheduling information such as date, time, and location (e.g., operating room number), attending physician(s), attending field agent(s), operation(s) to be performed, expected duration, patient information (e.g., biological details that may inform the parameters of the surgery, and thus the expected devices and products to be used), and the like. In one example of using such information to improve data coordination, the system may receive an identifier of the attending physician and an identifier of the operation to be performed, and select corresponding devices and products that the physician has approved (or eliminate devices the physician has rejected); the field agent may subsequently be limited to only the selected devices and products when creating the requisition order. In another example, the system may receive the patient information and operation identifier and use them to query the procedure data store or usage data store to determine an expected number of units of a particular product to be used in the operation; the field agent may subsequently be limited to selecting at most the expected number of units when creating the requisition order. Generally, creation of the requisition order may be subject to any of the limitations on type and quantity of devices and products to include, in accordance with the above descriptions.

Receiving the completed requisition order, the system may automatically transform the requisition order into the corresponding transaction forms for the facility (step 340) and the device provider (step 350). In one example, the system determines each product's description, quantity used, and price (i.e., agreed-upon price per unit and total price for all units), and injects the usage information into the order form used by the corresponding entity's accounting system. For example, the system uses the requisition order to identify the devices and products used in the agreed-upon pricing matrix, and creates the corresponding forms to include an appropriate order identifier (e.g., a purchase order number using the facility's format, and an invoice number using the device providers format), the device/product information, and the corresponding prices therefor. The forms may be delivered to the respective entities (i.e., the purchase order to the facility and the invoice to the device provider) via their user accounts, as described above, with an instruction for the entity to approve or make changes to the requisition. The system receives automated or manual approvals from the facility (step 360) and the device provider (step 370) and may correlate the approvals with the corresponding identifiers for each entity. For example, the system may generate a purchase order number according to the format used by the facility, and may generate an invoice number according to the format used by the device provider. This allows both entities to track the approved requisition in their respective local accounting systems, as well as on the platform. At step 380, after receiving both approvals the system may generate an invoice for the reconciled order of used devices and products, and deliver the invoice to the facility user account. The facility's accounting department pays this invoice to complete the surgical procedure.

Data generated throughout the methods performed by the system (e.g., method 300) may be aggregated (e.g., in a usage data store) as described above, and may be analyzed to provide insights into various operations at various points in the surgical procedure. For example, by tracking type and quantity of the devices and products used in a particular surgery, the system can establish an expected requisition and use it each time the surgery is performed. Combined with the agreed-upon pricing matrices between different pairs of device provider and facility, the system can estimate the cost of the surgery at different facilities and using different devices/products, and can thereby identify, for example, the lowest-cost option.

FIG. 4 illustrates an example matching algorithm used by the systems described herein to automatically correlate product offerings of a device provider with product needs of a medical facility, and then receive and process user input to produce a pricing agreement. Generally, a data coordination platform 402 as described above may obtain a product guide data structure(s) 462 describing the implant devices and associated surgical products provided by the device provider, and a pricing matrix data structure(s) 466 based on procedure and/or product pricing information provided by the facility, and may compare the data using stored (i.e., in memory) associations between data types of the data structures 462, 466 (e.g., product entries, procedure entries, anatomical categories, DRG codes, etc.) to produce match records 466 describing matches of offered products to needed products, and matches of requested prices to offered prices; the match records 466 may then be incorporated into a data structure 470 representing a pricing agreement between the device provider and the facility, and the pricing agreement data structure 470 may be stored (e.g., in a provider account data store 460, a facility account data store 464, and/or another data store of the platform 402) for later use in reconciling transactions.

In the illustrated example, an administrative user of the device provider may use a remote computing device 406 to access the platform 402 and upload a digital version of the product guide 410. A profile service 450 as described above, or another service of the platform 402, may transform the product guide 410 into the product guide data structure 462. Independently, an administrative user of the facility may use a remote computing device 408 to access the platform 402 and upload digital versions of one or more pricing matrices 412 used by the facility to organize offered prices for products used in various surgical procedures. The profile service 450 or another service may transform the pricing matrices 412 into the pricing matrix data structure 466. The data structures 462, 466 may be stored in the user account data 460, 464 of the respective user.

A matching service 452 of the platform 402 may receive the data structures 462, 466 and execute matching algorithms in accordance with one or more default and/or user-provided rule sets that define how similar the compared data between the pricing guide and the pricing matrix must be to constitute a match. The matching algorithms and rule sets may be stored in a matching data store 454 of the platform 402; the matching service 452 may obtain a suitable algorithm and the applicable rule set(s) from the data store 454 and perform the comparison to produce the match records 466, which may include only matches, or may also include non-matches, between the data structures 462, 466. The rule sets may include pricing rules. For example, a first pricing rule may specify that a product satisfying the requirements for a procedure is a match if the requested price (i.e., stated in the product guide) is no more than 1.15 times the offered price (i.e., stated in the facility's pricing matrix). Other example rules may apply to required documents and/or other product parameters; for example, the system may be instructed not to produce a match if a product with a material handling warning does not include a digitized spec sheet, or if a product that has been available for less than six months does not include a digitized copy of the FDA Section 510(k) clearance. Alternatively, the system may obtain the product guide data structure 462 and pricing matrix data structure(s) 466 and provide them to the device provider administrator in a user interface that enables the administrator to select a product entry from the product guide and electronically associate the selected product entry with a category, sub-category, and/or one or more procedures defined by the pricing matrix data structure(s); the matching service 452 may receive the selections and associations as user input, and process the user input to produce the match records 466.

The illustrated example shows the system producing three matches: the device providers price $X for product H, needed for procedure A, matches the price $X expected by the facility; the device provider's price $Z for product I, needed for procedure C, matches the price $Z expected by the facility; and, the device provider's price $N for product G, needed for the procedure B is within the threshold percentage T % set by the facility of the price $Y expected by the facility. The system may be configured to accept any price match (i.e., an exact match, or a mismatch within thresholds) automatically. Additionally, the system may be configured so that, at least for certain products (e.g., product I) and/or procedures (e.g., procedure C), a match of prices between the device provider and the facility does not automatically cause an acceptance by the facility and agreement to purchase the matched product. The system may present (e.g., via a user interface on the remote device 408) some or all of the price matches to an administrative user associated with the facility, and may enable the administrative user to enter user input accepting or declining to include the match in the pricing agreement. The illustrated example demonstrates the administrative user declining the price match for product I and accepting the other matches. The administrative user may be able to indicate prior to the execution of the matching algorithm that none, some, or all of the price matches can be automatically accepted. Once the feedback from the administrative user is received, the matching service 452 may create and store the pricing agreement data structure 470. Additionally or alternatively, the matching service 452 and/or other services of the platform 402 may place the pricing agreement into a “draft” state, and may enable the parties to correspond regarding a negotiation of the draft into a final pricing agreement; changes made during the negotiation may be entered by either party, and received and incorporated into the pricing agreement data structure 470 by services of the platform 402.

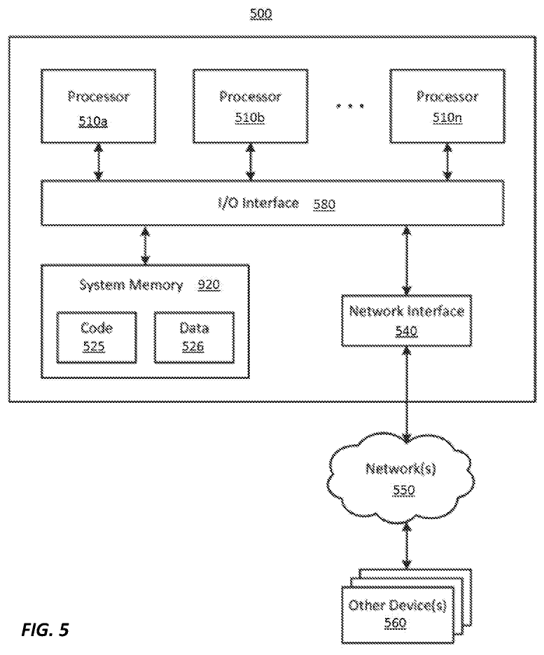

In at least some embodiments, a hardware computing device or group of interconnected hardware computing devices implementing a portion or all of one or more of the technologies described herein, including the techniques to implement a medical implant data coordination platform, can include a general- or special-purpose computer system that includes or is configured to access one or more computer-accessible media. FIG. 5 illustrates such (a) hardware computing device(s) 500. In the illustrated embodiment, computing device 500 includes one or more processors 510a, 510b, and/or 510n (which may be referred herein singularly as “a processor 510” or in the plural as “the processors 510”) coupled to a system memory 520 via an input/output (I/O) interface 580. Computing device 500 further includes a network interface 540 coupled to I/O interface 580.

In various embodiments, computing device 500 may be a uniprocessor system including one processor 510 or a multiprocessor system including several processors 510 (e.g., two, four, eight, or another suitable number). Processors 510 may be any suitable processors capable of executing instructions. For example, in various embodiments, processors 510 may be general-purpose or embedded processors implementing any of a variety of instruction set architectures (ISAs), such as the x86, Power PC, SP ARC, or MIPS ISAs, or any other suitable ISA. In multiprocessor systems, each of processors 510 may commonly, but not necessarily, implement the same ISA.

System memory 520 may be configured to store instructions and data accessible by processor(s) 510. In various embodiments, system memory 520 may be implemented using any suitable memory technology, such as static random access memory (SRAM), synchronous dynamic RAM (SDRAM), nonvolatile/Flash-type memory, or any other type of memory. In the illustrated embodiment, program instructions and data implementing one or more desired functions, such as those methods, techniques, and data described above, are shown stored within system memory 520 as code 525 and data 526.

In one embodiment, I/O interface 580 may be configured to coordinate I/O traffic between processor 510, system memory 520, and any peripheral devices in the device, including network interface 540 or other peripheral interfaces. In some embodiments, I/O interface 580 may perform any necessary protocol, timing, or other data transformations to convert data signals from one component (e.g., system memory 520) into a format suitable for use by another component (e.g., processor 510). In some embodiments, I/O interface 580 may include support for devices attached through various types of peripheral buses, such as a variant of the Peripheral Component Interconnect (PCI) bus standard or the Universal Serial Bus (USB) standard, for example. In some embodiments, the function of I/O interface 580 may be split into two or more separate components, such as a north bridge and a south bridge, for example. Also, in some embodiments some or all of the functionality of I/O interface 580, such as an interface to system memory 520, may be incorporated directly into processor 510.

Network interface 540 may be configured to allow data to be exchanged between computing device 500 and other device or devices 560 attached to a network or network(s) 550, such as other computer systems or devices as illustrated in FIG. 2, for example. In various embodiments, network interface 540 may support communication via any suitable wired or wireless general data networks, such as types of Ethernet networks, for example. Additionally, network interface 540 may support communication via telecommunications/telephony networks, such as analog voice networks or digital fiber communications networks, via storage area networks, such as Fiber Channel SANs or via any other suitable type of network and/or protocol.

In some embodiments, system memory 520 may be one embodiment of a computer-accessible medium configured to store program instructions and data for implementing embodiments of the present methods and apparatus. However, in other embodiments, program instructions and/or data may be received, sent or stored upon different types of computer-accessible media. Generally speaking, a computer-accessible medium may include non-transitory storage media or memory media, such as magnetic or optical media, e.g., disk or DVD/CD coupled to computing device 500 via I/O interface 580. A non-transitory computer-accessible storage medium may also include any volatile or non-volatile media, such as RAM (e.g., SDRAM, DDR SDRAM, RDRAM, SRAM, etc.), ROM, etc., that may be included in some embodiments of computing device 500 as system memory 520 or another type of memory. Further, a computer-accessible medium may include transmission media or signals such as electrical, electromagnetic or digital signals, conveyed via a communication medium such as a network and/or a wireless link, such as may be implemented via network interface 540. Portions or all of multiple computing devices, may be used to implement the described functionality in various embodiments; for example, software components running on a variety of different devices and servers may collaborate to provide the functionality. In some embodiments, portions of the described functionality may be implemented using storage devices, network devices, or special purpose computer systems, in addition to or instead of being implemented using general purpose computer systems. The term “computing device,” as used herein, refers to at least all these types of devices and is not limited to these types of devices.

Embodiments of the invention described and contemplated herein include, among others, a system including one or more hardware computing devices including a processor and memory storing program instructions that, when executed by the processor, cause the one or more hardware computing devices to provide, to a plurality of user computing devices in electronic communication with the one or more hardware computing devices over a network, a medical implant data coordination platform that: obtains a pricing agreement between a facility in which surgical procedures are performed and a device provider that provides a plurality of products used in the surgical procedures; receives user input data from an agent device of the plurality of user computing devices, the agent device associated with a field agent of the device provider; and, determines that the user input data comprises a requisition order describing usage of one or more of the plurality of products in a first surgical procedure. Further, the platform parses the requisition order to identify: the facility as the first surgical procedure's location; a first product, of the plurality of products, used in the first surgical procedure; and, a reported cost associated with the first product used in the first surgical procedure. The platform further: determines that the pricing agreement includes a first price limit describing an amount that the facility has agreed to pay the device provider for the first product; determines whether the requisition order is valid, wherein the requisition order is valid if the reported cost conforms to the first price limit; responsive to a determination that the requisition order is not valid, performs an action associated with rejecting the requisition order; and, responsive to a determination that the requisition order is valid, performs a transaction reconciliation process.

To perform the transaction reconciliation process, the platform may be configured to: generate, based on provider account information and the requisition order, a first invoice describing a payment due from the facility to the device provider for usage of one or more of the plurality of products in the first surgical procedure, the one or more of the plurality of products including the first product; send the first invoice to a provider computing device of the plurality of user computing devices, the provider computing device associated with the device provider; generate, based on facility account information and the requisition order, a second invoice describing costs associated with the first surgical procedure, the costs being due from a payor to the facility and including usage costs associated with the first product; and, send the second invoice to an accounting computing device of the plurality of user computing devices, the accounting computing device associated with the facility. To perform the transaction reconciliation process, the platform may be further configured to: generate, based on the facility account information and the requisition order, a draft purchase order describing the payment and the usage of the one or more of the plurality of products in the first surgical procedure; send the draft purchase order to the accounting computing device; receive a first approval message associated with the draft purchase order and indicating that the draft purchase order is approved; generate, based on the provider account information and the requisition order, a draft invoice describing the payment; send the draft invoice to the provider computing device; receive a second approval message associated with the draft invoice and indicating that the draft invoice is approved; and, to generate the first invoice, responsive to receiving the first approval message and the second approval message, transform the draft invoice using a provider invoice format to produce to the first invoice, the provider invoice format stored in the provider account information.

To obtain the pricing agreement, the platform may be configured to: receive procedure data representing a desired pricing matrix of the facility, the desired pricing matrix describing, for each of the surgical procedures, needed products used in the surgical procedure and one or more offered prices associated with the needed products; using a predetermined pricing matrix format, transform the procedure data to produce a pricing matrix data structure including a plurality of procedure definitions each describing one of the surgical procedures, each procedure definition including the one or more offered prices associated with the surgical procedure, and one or more of a plurality of product entries each describing one of the needed products; receive product data representing a product guide of the device provider, the product guide describing the plurality of products; using a predetermined product definition format associated with the pricing matrix format, transform the product data to produce a product guide data structure including a plurality of device entries each describing one of the plurality of products and comprising a product identifier and a requested price; determine a correlation of the product guide data structure to the pricing matrix data structure, the correlation identifying each of the plurality of products as one of the needed products, such that each of the device entries corresponds to one of the product entries; and, compare the plurality of device entries to the plurality of product entries to produce, as the pricing agreement, a pricing agreement data structure including a plurality of approved product entries each describing one of the products and an agreed-upon price limit, the plurality of approved product entries including a first approved product entry describing the first product and the first price limit.

The platform may be further configured to: parse the requisition order to further identify a reported quantity of the first product used in the first surgical procedure; and, determine a quantity limit representing proper usage of the first product in the first surgical procedure, wherein the requisition order is valid if the reported quantity conforms to the quantity limit.

Another embodiment of the invention described and contemplated herein is a system including an electronic user data store and one or more hardware computing devices in electronic communication with the user data store and including a processor and memory storing specific machine-readable instructions. The electronic data store stores provider account data associated with a device provider and comprising a product guide describing a plurality of products provided by the device provider for use in surgical procedures, and facility account data associated with a facility in which the surgical procedures are performed. The instructions, when executed by the processor, cause the one or more hardware computing devices to: receive, from an agent device associated with a field agent of a device provider and in communication with the one or more hardware computing devices via a computer network, information including a requisition order for a first product, of the plurality of products, used in a first surgical procedure performed at the facility; determine that the device provider is authorized to provide the first product at the facility; generate, based on the requisition order, the provider account data, and the facility account data, a plurality of transaction documents used by the device provider and the facility to reconcile a purchase of the first product in accordance with the requisition order; send at least one of the plurality of transaction documents to an accounting device associated with the facility; and, send at least one of the plurality of transaction documents to a provider device associated with the device provider.

The system may further include a first device client installed on the agent device and controlling communications between the agent device and the one or more hardware computing devices. The first device client may include program instructions executable by the agent device to: display, on a display of the agent device, a user interface enabling the field agent to identify the first product and to enter usage information associated with the first product and the first surgical procedure; receive user input data entered by the field agent using the user interface; and, send the user input data to the one or more hardware computing devices as the requisition order. The user interface may include one or more prompts for entering the usage information, the one or more prompts providing to the field agent a set of selectable values for one or more usage parameters, the set of selectable values being predetermined based on a procedure definition of the first surgical procedure. The one or more usage parameters may include a reported cost. The set of selectable values may include an agreed-upon price for the first product, the agreed-upon price being determined from a pricing agreement between the device provider and the facility. The user interface may be configured to prevent the field agent from entering a value for the reported cost other than the agreed-upon price.

The agent device may be a mobile device that generates geolocation information identifying a location of the agent device, and the device client or the one or more hardware computing devices may be further configured to: obtain the geolocation information; determine, based at least in part on the facility account data, a geographic boundary associated with the facility; determine, based on the geolocation information and the geographic boundary, whether the agent device is within the geographic boundary; responsive to a determination that the agent device is not within the geographic boundary, determine whether the field agent has entered the requisition order into the user interface; and responsive to a determination that the field agent has not entered the requisition order, cause the user interface to display a notification on the display of the agent device, the notification alerting the field agent to enter the requisition order.

The system may further include a second device client installed on a physician device in electronic communication, via the computer network, with the one or more hardware computing devices, the second device client controlling communications between the physician device and the one or more hardware computing devices. The second device client may include program instructions executable by the physician device to receive the requisition order and to enable a physician that performed the first surgical procedure to approve the requisition order.

The plurality of transaction documents may include: a draft purchase order describing the purchase and having a purchase order format used by the facility, the one or more hardware computing devices sending the draft purchase order to the accounting device for approval; a draft invoice describing the purchase and having an invoice format used by the device provider, the one or more hardware computing devices sending the draft invoice to the provider device for approval; a final purchase order generated based on a first approval associated with the draft purchase order, the one or more hardware computing devices sending the final purchase order to the provider device; and a final provider invoice generated based on one or both of the first approval and a second approval associated with the draft invoice, the one or more hardware computing devices sending the final provider invoice to the accounting device. The plurality of transaction documents may further include a final facility invoice generated based on procedure data describing all costs of the first surgical procedure to be paid by a payor to the facility the procedure data may include information associated with the purchase. The instructions, when executed, may further cause the one or more hardware computing devices to: generate, for the final purchase order, a purchase order number using the purchase order format; and generate, for the final provider invoice, an invoice number using the invoice format.

The instructions, when executed, may further cause the one or more hardware computing devices to: obtain a pricing agreement between the device provider and the facility; store the pricing agreement in the user data store as a pricing agreement data structure associated with the provider account data and the facility account data; and, prior to generating the plurality of transaction documents, compare the requisition order to the pricing agreement data structure to determine that the requisition order is valid under the pricing agreement. The provider account data describing the plurality of products may include a requested price associated with the first product. The facility account data describing the surgical procedures may include a price limit for a needed product used in the first surgical procedure. To obtain the pricing agreement, the instructions, when executed by the processor, may cause the one or more hardware computing devices to: determine, based on the provider account data describing the plurality of products and the facility account data describing the surgical procedures, that the first product corresponds to the needed product; determine that the requested price for the first product is a match for the price limit of the needed product; and create, in the pricing agreement data structure, a first product entry identifying the first product and an agreed-upon price, wherein the requisition order includes a reported cost of using the first product in the first surgical procedure, and wherein the requisition order is valid if the reported cost matches the agreed-upon price.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Finally, it is expressly contemplated that any of the processes or steps described herein may be combined, eliminated, or reordered. Accordingly, this description is meant to be taken only by way of example, and not to otherwise limit the scope of this invention.

What is claimed is:

1. A system, comprising one or more hardware computing devices including a processor and memory storing program instructions that, when executed by the processor, cause the one or more hardware computing devices to provide, to a plurality of user computing devices in electronic communication with the one or more hardware computing devices over a network, a medical implant data coordination platform that:
obtains a pricing agreement between a facility in which surgical procedures are performed and a device provider that provides a plurality of products used in the surgical procedures;
receives user input data from an agent device of the plurality of user computing devices, the agent device associated with a field agent of the device provider;
determines that the user input data comprises a requisition order describing usage of one or more of the plurality of products in a first surgical procedure;
parses the requisition order to identify:
the facility as the first surgical procedure's location;
a first product, of the plurality of products, used in the first surgical procedure; and
a reported cost associated with the first product used in the first surgical procedure;
determines that the pricing agreement includes a first price limit describing an amount that the facility has agreed to pay the device provider for the first product;
determines whether the requisition order is valid, wherein the requisition order is valid if the reported cost conforms to the first price limit;
responsive to a determination that the requisition order is not valid, performs an action associated with rejecting the requisition order; and
responsive to a determination that the requisition order is valid, performs a transaction reconciliation process.

2. The system of claim 1, wherein to perform the transaction reconciliation process, the platform is configured to:
generate, based on provider account information and the requisition order, a first invoice describing a payment due from the facility to the device provider for usage of one or more of the plurality of products in the first surgical procedure, the one or more of the plurality of products including the first product;
send the first invoice to a provider computing device of the plurality of user computing devices, the provider computing device associated with the device provider;
generate, based on facility account information and the requisition order, a second invoice describing costs associated with the first surgical procedure, the costs being due from a payor to the facility and including usage costs associated with the first product; and
send the second invoice to an accounting computing device of the plurality of user computing devices, the accounting computing device associated with the facility.

3. The system of claim 2, wherein to perform the transaction reconciliation process, the platform is configured to:
generate, based on the facility account information and the requisition order, a draft purchase order describing the payment and the usage of the one or more of the plurality of products in the first surgical procedure;
send the draft purchase order to the accounting computing device;
receive a first approval message associated with the draft purchase order and indicating that the draft purchase order is approved;
generate, based on the provider account information and the requisition order, a draft invoice describing the payment;
send the draft invoice to the provider computing device;
receive a second approval message associated with the draft invoice and indicating that the draft invoice is approved; and
to generate the first invoice, responsive to receiving the first approval message and the second approval message, transform the draft invoice using a provider invoice format to produce to the first invoice, the provider invoice format stored in the provider account information.

4. The system of claim 1, wherein to obtain the pricing agreement, the platform:
receives procedure data representing a desired pricing matrix of the facility, the desired pricing matrix describing, for each of the surgical procedures, needed products used in the surgical procedure and one or more offered prices associated with the needed products;
using a predetermined pricing matrix format, transforms the procedure data to produce a pricing matrix data structure comprising a plurality of procedure definitions each describing one of the surgical procedures and comprising:
the one or more offered prices associated with the surgical procedure; and
one or more of a plurality of product entries each describing one of the needed products;
receives product data representing a product guide of the device provider, the product guide describing the plurality of products;
using a predetermined product definition format associated with the pricing matrix format, transforms the product data to produce a product guide data structure comprising a plurality of device entries each describing one of the plurality of products and comprising a product identifier and a requested price;
determines a correlation of the product guide data structure to the pricing matrix data structure, the correlation identifying each of the plurality of products as one of the needed products, such that each of the device entries corresponds to one of the product entries;
compares the plurality of device entries to the plurality of product entries to produce, as the pricing agreement, a pricing agreement data structure comprising a plurality of approved product entries each describing one of the products and an agreed-upon price limit, the plurality of approved product entries including a first approved product entry describing the first product and the first price limit.

5. The system of claim 1, wherein the platform further:
parses the requisition order to further identify a reported quantity of the first product used in the first surgical procedure; and
determines a quantity limit representing proper usage of the first product in the first surgical procedure, wherein the requisition order is valid if the reported quantity conforms to the quantity limit.

6. A system, comprising:
an electronic user data store storing:
provider account data associated with a device provider and comprising a product guide describing a plurality of products provided by the device provider for use in surgical procedures; and
facility account data associated with a facility in which the surgical procedures are performed; and
one or more hardware computing devices in electronic communication with the user data store and including a processor and memory storing specific machine-readable instructions that, when executed by the processor, cause the one or more hardware computing devices to:
receive, from an agent device associated with a field agent of a device provider and in communication with the one or more hardware computing devices via a computer network, information comprising a requisition order for a first product, of the plurality of products, used in a first surgical procedure performed at the facility;
determine that the device provider is authorized to provide the first product at the facility;
generate, based on the requisition order, the provider account data, and the facility account data, a plurality of transaction documents used by the device provider and the facility to reconcile a purchase of the first product in accordance with the requisition order;
send at least one of the plurality of transaction documents to an accounting device associated with the facility; and
send at least one of the plurality of transaction documents to a provider device associated with the device provider.

7. The system of claim 6, further comprising a first device client installed on the agent device and controlling communications between the agent device and the one or more hardware computing devices, the first device client comprising program instructions executable by the agent device to:
display, on a display of the agent device, a user interface enabling the field agent to identify the first product and to enter usage information associated with the first product and the first surgical procedure;
receive user input data entered by the field agent using the user interface; and
send the user input data to the one or more hardware computing devices as the requisition order.

8. The system of claim 7, wherein the user interface comprises one or more prompts for entering the usage information, the one or more prompts providing to the field agent a set of selectable values for one or more usage parameters, the set of selectable values being predetermined based on a procedure definition of the first surgical procedure.

9. The system of claim 8, wherein:
the one or more usage parameters include a reported cost;
the set of selectable values includes an agreed-upon price for the first product, the agreed-upon price being determined from a pricing agreement between the device provider and the facility; and
the user interface prevents the field agent from entering a value for the reported cost other than the agreed-upon price.

10. The system of claim 7, wherein the agent device is a mobile device that generates geolocation information identifying a location of the agent device, and the device client or the one or more hardware computing devices is further configured to:
obtain the geolocation information;
determine, based at least in part on the facility account data, a geographic boundary associated with the facility;
determine, based on the geolocation information and the geographic boundary, whether the agent device is within the geographic boundary;
responsive to a determination that the agent device is not within the geographic boundary, determine whether the field agent has entered the requisition order into the user interface; and
responsive to a determination that the field agent has not entered the requisition order, cause the user interface to display a notification on the display of the agent device, the notification alerting the field agent to enter the requisition order.

11. The system of claim 7, further comprising a second device client installed on a physician device in electronic communication, via the computer network, with the one or more hardware computing devices, the second device client controlling communications between the physician device and the one or more hardware computing devices, the second device client comprising program instructions executable by the physician device to receive the requisition order and to enable a physician that performed the first surgical procedure to approve the requisition order.

12. The system of claim 6, wherein the plurality of transaction documents comprises:
a draft purchase order describing the purchase and having a purchase order format used by the facility, the one or more hardware computing devices sending the draft purchase order to the accounting device for approval;
a draft invoice describing the purchase and having an invoice format used by the device provider, the one or more hardware computing devices sending the draft invoice to the provider device for approval;
a final purchase order generated based on a first approval associated with the draft purchase order, the one or more hardware computing devices sending the final purchase order to the provider device; and
a final provider invoice generated based on one or both of the first approval and a second approval associated with the draft invoice, the one or more hardware computing devices sending the final provider invoice to the accounting device.

13. The system of claim 12, wherein the plurality of transaction documents further comprises a final facility invoice generated based on procedure data describing all costs of the first surgical procedure to be paid by a payor to the facility, the procedure data including information associated with the purchase.

14. The system of claim 12, wherein the instructions, when executed, cause the one or more hardware computing devices to:
generate, for the final purchase order, a purchase order number using the purchase order format; and
generate, for the final provider invoice, an invoice number using the invoice format.

15. The system of claim 6, wherein the instructions, when executed, further cause the one or more hardware computing devices to:
obtain a pricing agreement between the device provider and the facility;
store the pricing agreement in the user data store as a pricing agreement data structure associated with the provider account data and the facility account data; and
prior to generating the plurality of transaction documents, compare the requisition order to the pricing agreement data structure to determine that the requisition order is valid under the pricing agreement.

16. The system of claim 15, wherein:
the provider account data describing the plurality of products includes a requested price associated with the first product;
the facility account data describing the surgical procedures includes a price limit for a needed product used in the first surgical procedure; and
to obtain the pricing agreement, the instructions, when executed by the processor, cause the one or more hardware computing devices to:
determine, based on the provider account data describing the plurality of products and the facility account data describing the surgical procedures, that the first product corresponds to the needed product;
determine that the requested price for the first product is a match for the price limit of the needed product; and
create in the pricing agreement data structure a first product entry identifying the first product and an agreed-upon price, wherein the requisition order comprises a reported cost of using the first product in the first surgical procedure, and wherein the requisition order is valid if the reported cost matches the agreed-upon price.
